(19) United States
(12) Patent Application Publication
(10) Pub. No.: US 2011/0015537 A1
(43) Pub. Date: Jan. 20, 2011

(54) METHOD, APPARATUS AND COMPUTER PROGRAM FOR MONITORING SPECIFIC CEREBRAL ACTIVITY

(75) Inventors: Ville Henri Virkkala, Espoo (FI); Mika Olli Kristian Sarkela, Espoo (FI)

Correspondence Address: Andrus, Sceales, Starke & Sawall, LLP, 100 East Wisconsin Avenue, Suite 1100, Milwaukee, WI, 53202-4178, US

(73) Assignee: GENERAL ELECTRIC COMPANY, Schenectady, NY (US)

(21) Appl. No.: 12/503,187

(22) Filed: Jul. 15, 2009

(51) Int. Cl. A61B 5/0476

(52) U.S. Cl. 600/544

(57) ABSTRACT

A method, apparatus and computer program product are disclosed for monitoring specific cerebral activity of a subject. Occurrences of a predetermined event are detected in each of at least two brain wave signals acquired from a subject, wherein the predetermined event is indicative of a specific cerebral activity. Based on the detecting, an event information signal is produced for each of the at least two brain wave signals, thereby to obtain at least two event information signals, each event information signal being indicative of occurrences of the predetermined event in respective brain wave signal. Based on the at least two event information signals, an overall event information signal is generated, which is indicative of an occurrence of the predetermined event when at least one of the at least two event information signals is indicative of an occurrence of the predetermined event within a given time window, thereby to obtain a signal representing a general view of the specific cerebral activity of the subject within a measurement area covered by the at least two brain wave signals.

### BACKGROUND OF THE INVENTION

This disclosure relates generally to monitoring of cerebral activity of at least one type. This disclosure also relates to the determination of an activity index for at least one type of cerebral activity.

Brain function is conventionally measured by electroencephalography (EEG). Several electrodes are attached to the skull or scalp of a subject, thereby to obtain EEG signals on a plurality of measurement channels. The EEG signal information received from the subject is typically processed so that the signal of each measurement channel is processed independently of the signals of the other measurement channels, especially when a specific cerebral activity is of interest. Specific cerebral activity here refers to transient waveforms of a given type in the brain wave signal. The said waveforms have a characteristic morphology that differentiates the waveforms from continuous brain rhythms. Examples of such waveforms include epileptiform spikes and sharp waves, seizures, sleep spindles, K-complexes, microarousals, and bursts during burst-suppression activity. Below, the occurrence of a given transient waveform, such as an epileptiform spike, in a brain wave signal, i.e., on a measurement channel, is termed an ‘event’.

Current EEG monitoring methods aim to detect events using mostly single-channel EEG data, but concurrent appearance of the same event in the adjacent channels may also be utilized in the detection process. That is, in the detection stage, the decision whether or not an event is detected on a channel may be made based on the signal of that channel only or based on the signal of that channel and the signal(s) of one or more adjacent channels. Each waveform type is detected with a dedicated detector, i.e., if different types of events are to be detected, a dedicated detector is normally needed for each event type. After the detection stage, various parameters may be derived and monitored. For example, if epileptiform spikes are detected to monitor epileptiform activity, spike rate, also called spike density, may be determined for monitoring the degree of the epileptiform activity. If sleep spindles are detected to monitor sleep quality, a sleep quality measure or index may be produced by determining the rate/density of the sleep spindles, and if EEG bursts are detected in preterm infants, for example, inter-burst-interval may provide prognostic information. In the following, the parameters or measures that are determined based on the detected events are termed activity indexes. An activity index most often represents the rate or interval of a specific event, or the proportional duration of a specific event in a given time window.

The-above-mentioned, specific types of cerebral activities are often confined to certain area of the brain only. For example, sleep spindles are best visible in the central areas, whereas an optimal measurement site for seizures and epileptiform spikes cannot normally be anticipated, since the optimal site depends on the location of the damaged tissue. Also, the site of the most prominent appearance of seizures/spikes may vary during long-term monitoring. Therefore, the activity indexes provide accurate information only locally in a limited brain area, provided that a measurement site is near enough to the origin of the event. However, current trend in EEG monitoring is to use fewer electrodes, which often means that the origin of the waveform may be located relatively far from the measurement site. This complicates the detection of the events, since the ‘event-to-noise ratio’ decreases. As a result, one event may be detected on one channel but not on another channel, whereas the situation may be the opposite for another event. Assuming that the said events are manifestations of the same type of brain activity, it is obvious that the activity indexes derived from the two channels are inaccurate. Consequently, it is difficult to evaluate the overall activity level in the entire brain area that the measurement covers.

### BRIEF DESCRIPTION OF THE INVENTION

The above-mentioned problems are addressed herein which will be comprehended from the following specification.

In an embodiment, a method for monitoring specific cerebral activity of a subject comprises detecting occurrences of a predetermined event in each of at least two brain wave signals acquired from the subject, wherein the predetermined event is indicative of a specific cerebral activity, and producing, based on the detecting, an event information signal for each of the at least two brain wave signals, thereby to obtain at least two event information signals, each event information signal being indicative of occurrences of the predetermined event in respective brain wave signal. The method further includes generating, based on the at least two event information signals, an overall event information signal that indicates an occurrence of the predetermined event when at least one of the at least two event information signals is indicative of an occurrence of the predetermined event within a given time window, thereby to obtain a signal representing a general view of the specific cerebral activity of the subject within a measurement area covered by the at least two brain wave signals.

In another embodiment, an apparatus for monitoring specific cerebral activity of a subject comprises a first detector unit configured to detect occurrences of a predetermined event in each of at least two brain wave signals acquired from a subject, wherein the predetermined event is indicative of a specific cerebral activity, and a second detector unit, responsive to first detector unit and configured to produce an event information signal for each of the at least two brain wave signals, thereby to obtain at least two event information signals, each event information signal being indicative of detected occurrences of the predetermined event in respective brain wave signal. The apparatus further includes a first determination unit configured to generate, based on the at least two event information signals, an overall event information signal that indicates an occurrence of the predetermined event when at least one of the at least two event information signals is indicative of an occurrence of the predetermined event within a given time window, thereby to obtain a signal representing a general view of the specific cerebral activity of the subject within a measurement area covered by the at least two brain wave signals.

In a still further embodiment, a computer program product for monitoring specific cerebral activity of a subject comprises a first program product portion configured to generate an overall event information signal indicative of an occurrence of a predetermined event when at least one of at least two event information signals is indicative of an occurrence of the predetermined event within a given time window, wherein the predetermined event is indicative of a specific cerebral activity of the subject and each of the at least two event information signals is indicative of detected occurrences of the predetermined event in respective brain wave signal acquired from the subject, the overall event information signal thus providing a general view of a specific cerebral activity of the subject.

Various other features, objects, and advantages of the invention will be made apparent to those skilled in the art from the following detailed description and accompanying drawings.

### BRIEF DESCRIPTION OF THE DRAWINGS

FIG. 1 is a block diagram illustrating one embodiment of a mechanism for monitoring specific cerebral activity;

FIG. 2 illustrates an example of overall event rate calculation;

FIG. 3 illustrates an example of overall event ratio calculation;

FIG. 4 is a block diagram illustrating the detection and index determination stages in one embodiment of the method/apparatus;

FIG. 5 illustrates an example of the overall activity index and three channel-specific activity indexes measured from a subject;

FIG. 6 illustrates an embodiment of the apparatus for monitoring at least one type of cerebral activity; and

FIG. 7 is a block diagram illustrating the detection and index determination stages in a further embodiment of the apparatus.

### DETAILED DESCRIPTION OF THE INVENTION

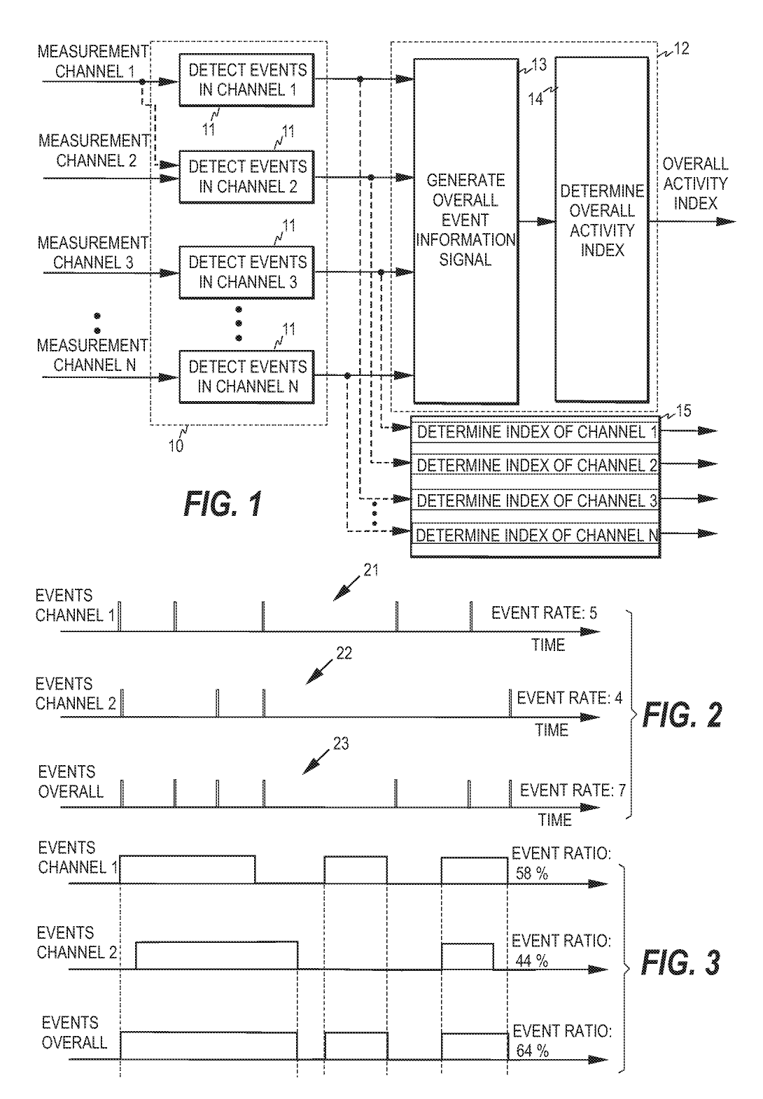

FIG. 1 illustrates one embodiment of the detection and index determination stages of an apparatus for monitoring a specific type of cerebral activity in a subject. It is assumed here that the measurement setup includes N (N≧2) measurement channels. That is, it is assumed that N brain wave signals, such as EEG signals, are obtained from the subject. For detecting specific events, i.e., occurrences of a specific transient waveform, in the brain wave signal, each measurement channel is provided with a detector 11 designed to detect such an event (i.e., the specific waveform). In each detector, the detection of the events may be carried out based on the respective brain wave signal only, or based on the respective brain wave signal and the brain wave signal of one or more of the adjacent measurement channels, as is illustrated in case of channel 2 in the figure. Each detector thus outputs a signal that is indicative of the occurrences of the event in the input signal of the detector. The output signals of the detectors are therefore termed event information signals in this context. In a typical application, the event information signals are time-series signals. In one embodiment, the output signals of the detectors have only two values, a logic “true” indicating that the specific waveform is currently present in the input signal and a logic “false” indicating that the specific waveform is currently not present in the input signal. These binary logic embodiments are discussed first.

Each event information signal is supplied to an overall index determination unit 12 that comprises two stages in this embodiment. In the first stage 13, an overall event information signal is generated from the event information signals of the measurement channels. The overall event information signal is a signal that is indicative of the occurrence of a single event whenever an event is detected in at least one of the brain wave signals. The overall event information signal therefore provides a general view of the appearance of the specific waveform in the entire measurement area covered by the N measurement channels, typically in the entire brain. The overall event information signal indicates absence of events only when an event is not detected in any of the channel-specific signals. Consequently, if an event is detected substantially simultaneously in more than one brain wave signal (channel), the overall event information is indicative of a single event. The same is true when an event is detected in only one brain wave signal. The overall event information signal may be produced, for example, by applying a Boolean OR operation to the channel-specific event information signals.

In addition to the overall event information signal produced in the first stage 13, an overall activity index indicative of the degree of the specific activity in the brain may be produced based on the overall event information signal in the second stage 14 of unit 12. A channel-specific index may further be derived from one or more of the channel-specific event information signals, as the dashed arrows indicate in the figure. The channel-specific activity indexes are determined in unit 15.

FIG. 2 illustrates an example of the operation of the overall index determination unit 12 when the activity index is indicative of event rate, such as spike rate. The figure shows examples of the event information signals 21, 22 of two measurement channels (here, N=2) and of an overall event information signal 23 produced in stage 13 based on the two channel-specific event information signals 21 and 22.

In stage 13, the overall event information signal is generated by generating a single event (i.e., a signal value indicative of an event) whenever an event occurs on either of the two channels. A single event is also generated if an event occurs in both (all) channels substantially simultaneously, i.e., within a given (short) time window termed time slot in this context. The overall event information signal 23 may be produced, for example, by using the data sequences of the event information signals 21, 22 as the operands of a Boolean OR operation. In the example shown, the event rates of the measurement channels are 5 (channel 1) and 4 (channel 2), and the event rate of the overall signal 23 is thus seven (seven events in the time window shown). In stage 14, the overall activity index may be determined by counting the number of events in each successive measurement window. The length of the measurement window may depend on the activity type monitored. In case of spikes, the length of measurement window may be, for example, 1 minute.

FIG. 3 illustrates an example of the same signals when the activity index is indicative of an event ratio, i.e., the ratio of event duration to the total time. The lengths of the pulses indicate the event duration periods. In stage 13, the overall event information signal is generated by starting an event (logic “true” value) when an event is detected in any of the channels and ending the event when the same event is not anymore detected in any of the channels. In stage 14, the ratio of the duration of the logic “true” to the length of the measurement window is determined. In the example of FIG. 3, the overall index value is 64%, while the channel-specific values are 58% and 44%. Typical examples of this type of indexes are burst suppression ratio and seizure ratio.

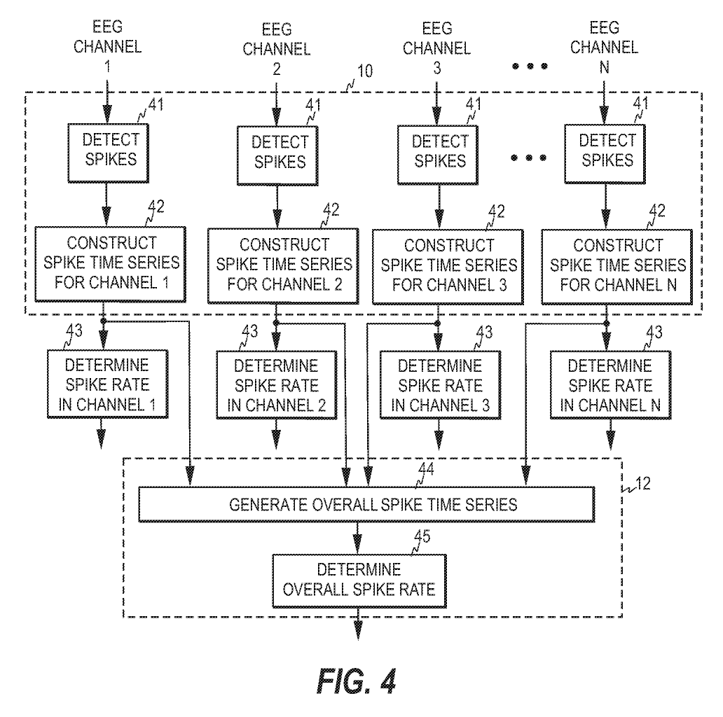

FIG. 4 illustrates the detection and index determination stages in one embodiment of a method intended for detecting brain wave spikes, such as EEG spikes. It is again assumed that the brain wave signals are received from N measurement channels. Each channel is provided with a spike detector that detects spikes in the signal waveform (steps 41). Based on the detected spikes, a spike time series is constructed in steps 42 for each measurement channel. This time series corresponds to the event information signals 21, 22 of FIG. 2. Each event information signal is supplied to a channel-specific spike rate unit, in which the spike rate of the channel is determined (steps 43), and to the overall index determination unit 12, where the spike time series, i.e., the event information signals, of the measurement channels are combined to generate an overall spike time series (step 44). This may be carried out as is illustrated in connection with FIG. 2. The overall spike rate is determined from the overall spike time series by counting the number of spikes in consecutive measurement windows of the time series (step 45), thereby to obtain the overall spike rate, i.e., the overall activity index.

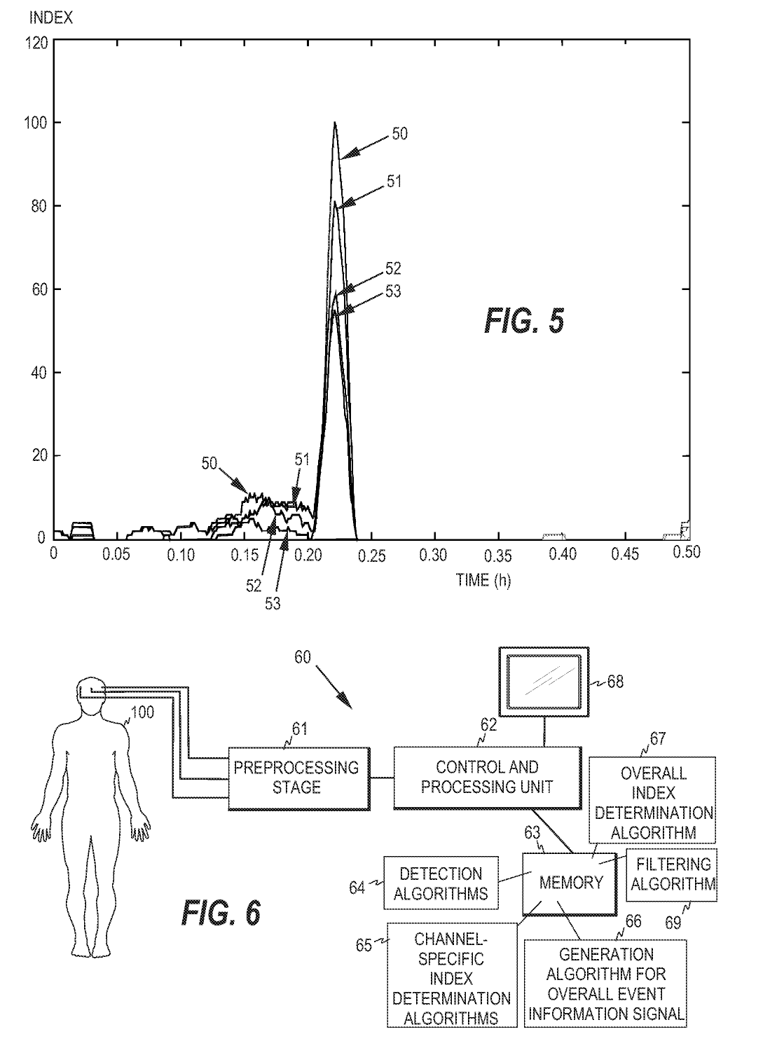

FIG. 5 shows the channel-specific spike rates and the overall spike rate measured from a subject. Curve 50 shows the overall spike rate (overall activity index) determined in the above-described manner, curve 51 shows the spike rate measured from left frontal EEG channel, curve 52 the spike rate measured from interhemispheric frontal EEG channel, and curve 53 the spike rate measured from right-frontal EEG channel. By determining the overall activity index and the channel-specific activity indexes and presenting them graphically as in FIG. 5, it is easy to see how wide-spread the dysfunction is by comparing the heights of the curves of the individual channels to the height of overall activity index curve. FIG. 5 indicates, for example, that more than half of all events appear on all three channels, since all channel-specific activity indexes are at least half of the overall activity index.

In the above embodiments, all event information signals represent traditional binary logic, in which an event is either present or not present, i.e., the event information signals (output signals of detectors) comprise only “true” and “false” values. However, multi-valued logic that allows more than two truth values may also be used. Fuzzy logic and Bayesian logic are examples of such logics. When fuzzy logic is used, the sequences of the event information signals could be, for example, as shown in Table 1 below.

TABLE 1

| | Time | Time | Time | Time | Time | Time | Time |
|---|---|---|---|---|---|---|---|
| | slot 1 | slot 2 | slot 3 | slot 4 | slot 5 | slot 6 | slot 7 |
| Channel 1 | 0 | 0.6 | 0 | 0 | 0.9 | 0 | 0 |
| Channel 2 | 0 | 0.8 | 0 | 0 | 0.5 | 0 | 0.3 |
| Overall | 0 | 0.8 | 0 | 0 | 0.9 | 0 | 0.3 |

As discussed above, the overall event information signal is a signal that is indicative of the occurrence of a single event whenever an event is detected in at least one of the brain wave signals. In fuzzy logic, the signal values are membership values indicating the degree of truth of an event. Table 1 illustrates a two-channel example, in which the first row shows a sequence of the event information signal of channel 1, the second row the concurrent sequence of the event information signal of channel 2, and the third row the resulting sequence of the overall event information signal. In each time slot, the value of the signal represents the membership of an event. For example, a detector outputs a value 0.5 if it decides that the membership of an event in the time slot is 0.5. The time series of the overall event information signal is in this example generated by applying MAX operation on the channel-specific event information signals in each time slot, i.e., in each time slot the overall event information signal obtains a value that is equal to the highest channel-specific membership value in that time slot.

In a further embodiment, a predetermined criterion may be set, which determines the set of event information signal values that are regarded as valid. For example, the predetermined criteria may dictate that an event is regarded as a valid event only when the associated membership value is greater than 0.5. If this criterion is applied to the example of Table 1, the overall event information signal sequence is otherwise the same, but the last value is converted to zero, since the said value is below 0.5. The filtering out of the values that are regarded as invalid may be carried out in the detectors or after the detector but before the channel-specific indexes are determined in unit 15. That is, if the filtering is carried out, the same filtered values serve as input signals for units 12 and 15. Each activity index may be determined by adding the signal values of the respective event information signal in each consecutive measurement window. Alternatively, depending on the used logic, probability or likelihood values of an event may be used instead of membership values. Membership, probability, and likelihood values all represent the degree of credibility of the detected event. This kind of property may be incorporated into the detection algorithm. In this context, values describing the credibility are called credibility values.

EEG monitoring of long-term neurological patients allows the effects of the treatment, such as medication, to be tracked. This will probably improve the outcome in some patient groups whereas in some other patient groups earlier indication of poor prognosis is obtained. It is well known that inter-observer agreement in spike analysis is poor. However, by examining overall epileptiform brain activity (using wider time scales and larger brain areas) instead of single spikes almost perfect agreement between experts has been reported (Wilson et al. Spike detection. Correlation and reliability of human experts. Electroenceph Clin Neurophys, 98:186-198, 1996). The overall event information signal and the associated overall index offer an efficient tool for estimating the overall level of brain dysfunction. This tool is particularly efficient when the ability of the individual channels to detect all events is reduced, due to the low number of electrodes used. Furthermore, the comparison of the overall activity index with the channel-specific indexes provides useful information about the distribution of the dysfunction within the brain.

FIG. 6 illustrates one embodiment of a monitoring apparatus 60 for monitoring at least one type of cerebral activity in a subject 100. As discussed above, the number of electrodes attached to the skull or scalp of the subject may vary. However, the number of brain wave signals obtained from the measurement channels is at least two. The measured brain wave signals are supplied to a control and processing unit 62 through a preprocessing stage 61 that may include normal preprocessing functions, such as amplification and/or artifact removal. The control and processing unit converts the signals into digitized format for each measurement channel. The digitized signal data may then be stored in the memory 63 of the control and processing unit. The digitized signal data may be utilized by detection algorithms 64 adapted to detect, when executed by the control and processing unit, the events on each measurement channel and to produce the event information signal for each channel. The resulting event information sequences may be stored in the memory of the control and processing unit. The sequences may comprise only two logic values (true/false) or credibility values between 0 and 1.

The control and processing unit is further provided with an algorithm 66 for generating the overall event information time series based on the multiple channel-specific event information time series. As discussed above, this algorithm produces a single event when an event is detected in one or more of the channel-specific event information signals. The single event may be indicated as a logic “true” value or as a credibility value derived from the concurrent channel-specific credibility values.

The control and processing unit may further be provided with respective algorithms 65 and 67 adapted to determine, when executed by the control and processing unit, the channel-specific activity indexes based on the channel-specific event information signals and the overall activity index based on the overall event information signal.

The filtering algorithms configured to “remove” signal values regarded as invalid may be separate algorithms 69 stored in memory 63 or algorithms that are integrated with the detection algorithms 64.

The control and processing unit is further configured to control the display unit 68 of the apparatus for displaying the results to the user of the apparatus. As discussed above, it is not necessary to display the index values, but only the overall event information signal and possibly also the channel-specific event information signals may be displayed to the user, since the user may deduce the degree of the overall activity based on the overall event information signal only. However, the determination of the overall activity index is useful whenever the interpretation of the graphical presentation of the overall event information signal is not straightforward.

In terms of the determination of the overall activity information, the control and processing unit, which is adapted to execute the above-described algorithms, may thus be seen as an entity of at least two operational modules or units, as is illustrated in FIG. 1. A detector unit 10 is configured to detect the events in the brain wave signals and to produce the channel-specific event information signals, and a first determination unit 13 is configured to generate the overall event information signal. In terms of the index determination, two additional operational units may also be provided: a second determination unit 14 configured to generate the overall activity index based on the overall event information signal and a third determination unit 15 configured to determine a plurality of channel-specific activity indexes. The monitoring thus utilizes the output signals of common detectors to produce an “overall output signal” that reflects the specific activity in the entire measurement area and thus provides a general view of the specific type of activity.

As may be deduced from the above description of FIG. 6, a conventional EEG monitor that comprises units 10 and 15, i.e., algorithms 64 and 65, may be upgraded to enable the device to determine the overall event information signal and possibly also the overall activity index in the above-described manner. Such an upgrade may be implemented, for example, by delivering to the device a software module that enables the device to utilize the output signals of the detectors in the above-described manner. The software module is therefore configured to perform, when executed by the control and processing unit, the operations corresponding to algorithm 66 and possibly also those of algorithm 67. The software module may be delivered, for example, on a data carrier, such as a CD or a memory card, or the through a telecommunications network. The software module may further include a filtering unit for filtering out invalid values of the event information signals (algorithm 69).

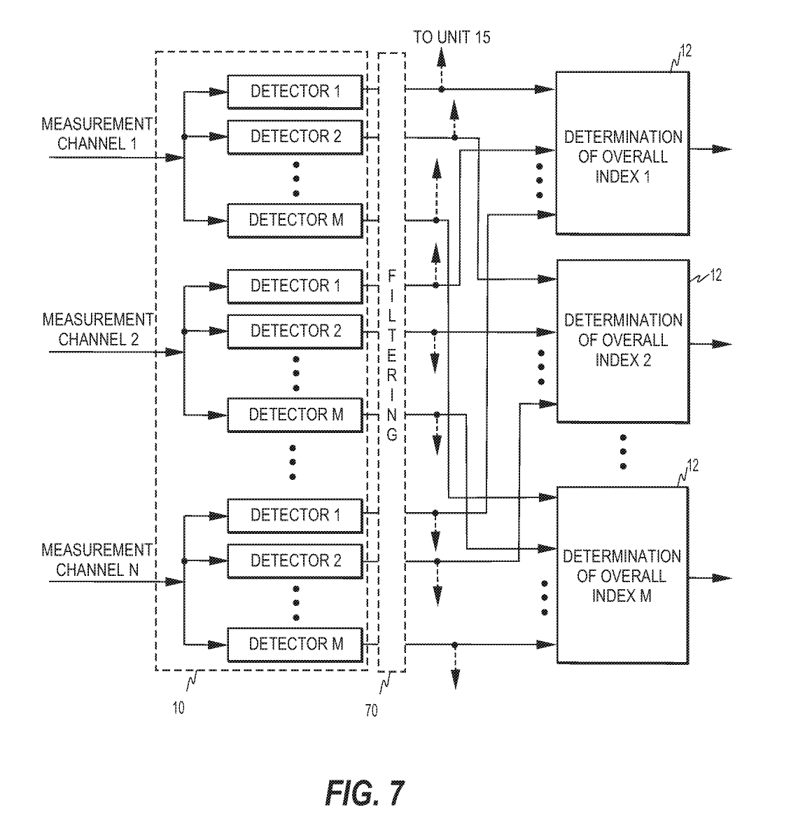

The patient monitor may also be equipped to detect different types of events on each measurement channel. This may be implemented by providing each measurement channel with M (M≧2) parallel detectors, as is shown in FIG. 7. Each of the M detectors is designed to detect the occurrences of a specific event in the respective brain wave signal, i.e., the apparatus can detect M different types of events (waveforms). The apparatus therefore comprises M determination units 12, each generating the overall event information signal, and possibly also the activity index, for the respective event type. Thus, the device may output M overall activity indexes. The dashed arrows illustrate that the individual event information signals may be connected to determination unit 15 (not shown) configured to determine channel-specific activity indexes for each event type. In this embodiment, M overall activity indexes and M×N channel-specific activity indexes are thus obtained. However, certain event types may be detected on selected channels only. The figure also shows an optional filtering unit 70, in which invalid signal values may be converted to a predetermined value, such as zero. As discussed above, the filtering unit comprises two operational entities; an examination unit configured to examine whether the credibility values fulfill a predetermined criterion and a conversion unit configured to convert the credibility values that fulfill the criterion.

In the above examples, the detection is carried out for each brain wave signal acquired from the subject. This is not necessarily the case in all embodiments, but the number of brain wave signals acquired from the subject may be greater than the number of brain wave signals for which event detection is carried out. That is, the measurement set-up may include auxiliary channels on which events are not detected.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural or operational elements that do not differ from the literal language of the claims, or if they have structural or operational elements with insubstantial differences from the literal language of the claims.

1. A method for monitoring specific cerebral activity of a subject, the method comprising:
detecting occurrences of a predetermined event in each of at least two brain wave signals acquired from a subject, wherein the predetermined event is indicative of a specific cerebral activity;
producing, based on the detecting, an event information signal for each of the at least two brain wave signals, thereby to obtain at least two event information signals, each event information signal being indicative of occurrences of the predetermined event in respective brain wave signal; and
generating, based on the at least two event information signals, an overall event information signal that indicates an occurrence of the predetermined event when at least one of the at least two event information signals is indicative of an occurrence of the predetermined event within a given time window, thereby to obtain a signal representing a general view of the specific cerebral activity of the subject within a measurement area covered by the at least two brain wave signals.

2. The method according to claim 1, further comprising determining an overall activity index based on the overall event information signal, wherein the overall activity index is indicative of a degree of occurrences of the predetermined event, said occurrences being indicated by the overall event information signal.

3. The method according to claim 1, wherein the producing includes producing the at least two event information signals, in which each event information signal assumes a logic true value when an occurrence of the predetermined event is detected and a logic false value otherwise.

4. The method according to claim 3, wherein the generating includes applying a logical OR operation to the at least two event information signals, thereby to obtain the overall event information signal.

5. The method according to claim 1, wherein the producing includes producing the at least two event information signals, in which each event information signal assumes credibility values indicative of credibility of the occurrences of the predetermined event.

6. The method according to claim 5, wherein the generating includes applying a MAX operation to the at least two event information signals, thereby to obtain the overall event information signal.

7. The method according to claim 5, further comprising
examining whether the credibility values fulfill a predetermined criterion; and
converting the credibility values that fulfill the predetermined criterion to a predetermined value.

8. The method according to claim 1, wherein
the detecting further includes detecting at least one further predetermined event in each of the at least two brain wave signals;
the producing further includes producing at least two further event information signals for each of the at least one further predetermined event; and
the generating further includes generating a dedicated overall event information signal for each of the at least one further predetermined event, thereby to obtain at least one further overall event information signal in addition to the overall event information signal.

9. The method according to claim 2, wherein the determining comprises determining the overall index, in which the overall index is indicative of one variable in a set of variables comprising rate and proportional duration of the predetermined event.

10. An apparatus for monitoring specific cerebral activity of a subject, the apparatus comprising:
a first detector unit configured to detect occurrences of a predetermined event in each of at least two brain wave signals acquired from a subject, wherein the predetermined event is indicative of a specific cerebral activity;
a second detector unit, responsive to first detector unit and configured to produce an event information signal for each of the at least two brain wave signals, thereby to obtain at least two event information signals, each event information signal being indicative of detected occurrences of the predetermined event in respective brain wave signal; and
a first determination unit configured to generate, based on the at least two event information signals, an overall event information signal that indicates an occurrence of the predetermined event when at least one of the at least two event information signals is indicative of an occurrence of the predetermined event within a given time window, thereby to obtain a signal representing a general view of the specific cerebral activity of the subject within a measurement area covered by the at least two brain wave signals.

11. The apparatus according to claim 10, further comprising a second determination unit configured to determine an overall activity index based on the overall event information signal, wherein the overall activity index is indicative of a degree of occurrences of the predetermined event, said occurrences being indicated by the overall event information signal.

12. The apparatus according to claim 10, wherein each event information signal assumes a logic true value when an occurrence of the predetermined event is detected and a logic false value otherwise.

13. The apparatus according to claim 12, wherein the first determination unit is configured to apply a logical OR operation to the at least two event information signals, thereby to obtain the overall event information signal.

14. The apparatus according to claim 10, wherein each event information signal assumes credibility values indicative of credibility of the occurrences of the predetermined event.

15. The apparatus according to claim 14, wherein the first determination unit is configured to apply a MAX operation to the at least two event information signals, thereby to obtain the overall event information signal.

16. The apparatus according to claim 14, further comprising:
an examination unit configured to examine whether the credibility values fulfill a predetermined criterion; and
a conversion unit configured to convert the credibility values that fulfill the predetermined criterion.

17. The apparatus according to claim 10, wherein
the first detector unit is further configured to detect at least one further predetermined event in each of the at least two brain wave signals;
the second detector unit is further configured to produce at least two further event information signals for each of the at least one further predetermined event; and
the first determination unit is further configured to generate a dedicated overall event information signal for each of the at least one further predetermined event, thereby to obtain at least one further overall event information signal in addition to the overall event information signal.

18. The apparatus according to claim 11, wherein the overall index is indicative of one variable in a set of variables comprising rate and proportional duration of the predetermined event.

19. A computer program product for monitoring specific cerebral activity of a subject, the computer program product comprising a first program product portion configured to generate an overall event information signal indicative of an occurrence of a predetermined event when at least one of at least two event information signals is indicative of an occurrence of the predetermined event within a given time window, wherein the predetermined event is indicative of a specific cerebral activity of a subject and each of the at least two event information signals is indicative of detected occurrences of the predetermined event in respective brain wave signal acquired from the subject, the overall event information signal thus providing a general view of a specific cerebral activity of the subject.

20. The computer program product according to claim 19, further comprising a second program product portion configured to determine an overall activity index based on the overall event information signal, wherein the overall activity index is indicative of a degree of occurrences of the predetermined event, said occurrences being indicated by the overall event information signal.
